(19) United States
(12) Patent Application Publication
(10) Pub. No.: US 2012/0130265 A1
(43) Pub. Date: May 24, 2012

(54) Portable Spirometer

(76) Inventors: Un Jong Cha, Chungbuk (KR); Kyung-Ah Kim, Chungbuk (KR); In-Kwang Lee, Chungbuk (KR); Sung-Sik Kim, Gyeonggi-do (KR)

(21) Appl. No.: 13/318,285

(22) PCT Filed: Oct. 19, 2010
(86) PCT No.: PCT/KR10/07144

§ 371 Date: Jan. 13, 2012

(30) Foreign Application Priority Data
Dec. 24, 2009 (KR) 10-2009-0130470

(51) Int. Cl. A61B 5/087

(52) U.S. Cl. 600/538

(57) ABSTRACT

Disclosed is a portable spirometer which comprises: a small breath tube for measuring a unidirectional flow, to which the breath flow of a patient is inputted; a breath signal processing unit which generates a breath signal from the breath flow, removes the noise contained in the breath signal and amplifies the signal level so as to generate a target signal of analysis; a breath signal analysis unit for analyzing the target signal of analysis to calculate diagnosis parameters; and a display unit for displaying the analysis result of the breath signal.

### FIELD

The present invention relates to a portable spirometer. The portable spirometer may have a portable structure, and may correspond to a breath flow measuring device capable of measuring and analyzing lung capacity.

### BACKGROUND

Measuring of lung capacity in a breath test and measuring of a heartbeat of a patient may provide useful information for diagnosing whether a patient has a breathing disorder and a cardiac disorder, for example, myocardial infarction, atrial fibrillation, and the like.

A scheme of measuring lung capacity may be classified into a type corresponding to a scheme of directly measuring a variation of a lung volume while a patient is breathing and a type corresponding to a breath flow measuring scheme of detecting and measuring a flow flowing in and out of a lung while a patient is breathing.

Conventionally, the scheme of directly measuring a variation of a lung volume has been primarily used in measuring lung capacity. However, the breath flow measuring scheme is being used more frequently.

A conventional breath flow measuring device such as a clinical spirometer may be manufactured for clinical use and thus, may be high-priced and big in size, which may prevent people with chronic respiratory problems from easily measuring a breath flow by carrying the device. Through miniaturization of an electronic spirometer to achieve portability, it may be difficult to miniaturize a sensor device for measuring a breath flow that converts a directly immeasurable living body variable into a measurable physical variable.

A conventional pneumotachograph may be difficult to be miniaturized since a fluid resistance may be inserted into a breath path (a breath tube), and a structure of the fluid resistance including a mesh screen, a capillary tube, and the like may be inappropriate to miniaturization. A tubinometer may be difficult to be miniaturized since a rotating turbine may be included on a breath path (a breath tube).

### DETAILED DESCRIPTION

### Technical Goals

An aspect of the present invention provides a portable spirometer that may be easily carried and is capable of easily measuring a breath flow.

Another aspect of the present invention provides a portable spirometer that may be used for a medical treatment and a telemedicine.

Still another aspect of the present invention provides a portable spirometer that may expend a relatively low amount of power and effectively perform wired and wireless communication.

Technical Solutions

According to an aspect of the present invention, there is provided a portable spirometer, including a small breathing tube for measuring a unidirectional flow, to which a breath flow of a patient is inputted, a breath signal processing unit to generate a breath signal from the breath flow, remove noise contained in the breath signal, and amplify a signal level so as to generate a target signal for analysis, a breath signal analysis unit to analyze the target signal for analysis to calculate a diagnosis parameter, and a display unit to display an analysis result of the breath signal.

The small breathing tube for measuring a unidirectional flow may include a circular tube including an entrance, formed by disposable paper or plastic, to be brought into contact with the mouth of the patient, and an outlet opposing the entrance, and a sensing path formed to be adjacent to the outlet of the circular tube and to pass through the circular tube from an upper portion to be extended to a lower portion outside of the circular tube, and formed to have a tubular shape in which the upper portion is closed and the lower portion is open.

The sensing path may have multiple sampling holes for measuring a flow separated by a predetermined interval along a lengthwise direction at an entrance side of the circular tube, on a breathing route of the circular tube.

The portable spirometer may further include a power controller to block a power supply to the breath signal processing unit and the breath signal analysis unit in response to the small breathing tube for measuring a unidirectional flow being removed.

The portable spirometer may further include a wireless communication unit to wired-exchange data with an external device, a wired communication unit to wiredly exchange data with the external device, and a communication mode selector to inactivate one of the wireless communication unit and the wired communication unit in response to the same device being connected to the wireless communication unit and the wired communication unit.

The portable spirometer may further include a connection controller to inactivate the breath signal analysis unit, and control so that the target signal for analysis does not pass through the breath signal analysis unit and is transmitted to the external device through the wireless communication unit or the wired communication unit in response to the wireless communication unit or the wired communication unit being connected to the external device.

The portable spirometer may further include a storage unit to store the analysis result of the breath signal, wherein the display unit displays data corresponding to a highest lung capacity measurement value in data stored in the storage unit in response to power being turned ON.

The diagnosis parameter may include at least one of a peak expiratory flow rate (PEF), a first second forced expiratory volume (FEV 1.0), a forced vital capacity (FVC), and FEV 1.0/FVC.

### Effect of the Invention

According to embodiments of the present invention, it is possible to provide a portable spirometer that may be easily carried and is capable of easily measuring a breath flow.

According to embodiments of the present invention, it is possible to provide a portable spirometer that may be used for a medical treatment and a telemedicine.

According to embodiments of the present invention, it is possible to provide a portable spirometer that may expend a relatively low amount of power and effectively perform wired and wireless communication.

### BRIEF DESCRIPTION OF DRAWINGS

FIG. 1 illustrates a configuration of a portable spirometer according to an embodiment of the present invention.

FIG. 2 illustrates a configuration of a portable spirometer according to another embodiment of the present invention.

FIG. 3 illustrates a cross-sectional view of a small breath tube, for measuring a unidirectional flow, of FIG. 1 or FIG. 2.

### EMBODIMENTS OF THE INVENTION

Reference will now be made in detail to embodiments of the present invention, examples of which are illustrated in the accompanying drawings, wherein like reference numerals refer to the like elements throughout. The embodiments are described below in order to explain the present invention by referring to the figures.

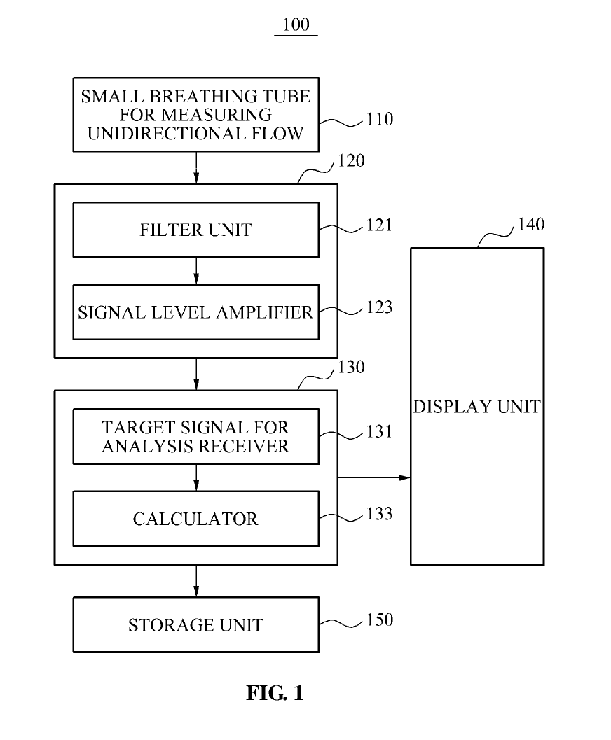

FIG. 1 illustrates a configuration of a portable spirometer according to an embodiment of the present invention.

Referring to FIG. 1, a portable spirometer 100 may include a small breathing tube for measuring a unidirectional flow 110, a breath signal processing unit 120, a breath signal analysis unit 130, and a display unit 140. The portable spirometer 100 may further include a storage unit 150.

The small breathing tube for measuring a unidirectional flow 110 may receive an input of a breath flow of a patient. The small breathing tube for measuring a unidirectional flow 110 may be detachable, and may be formed by disposable paper or plastic. The small breathing tube for measuring a unidirectional flow 110 may further include a breath detection sensor (not shown). In this instance, the breath detection sensor may detect a temperature or a pressure of the breath flow, and generate a breath flow signal.

The breath signal processing unit 120 may generate a breath signal from the breath flow or the breath flow signal, remove noise contained in the breath signal, and amplify a signal level so as to generate a target signal for analysis. The breath signal processing unit 120 may include a filter unit 121 and a signal level amplifier 123. The filter unit 121 may remove noise contained in the breath signal, and the signal level amplifier 123 may amplify a signal level of the breath signal from which noise is removed. The breath signal, from which noise is removed, having an amplified signal level may correspond to the target signal for analysis.

According to an embodiment, the breath signal processing unit 120 may further include a differential pressure sensor (not shown) to generate an electric signal by detecting a dynamic pressure.

The breath signal analysis unit 130 may analyze the target signal for analysis to calculate a diagnostic parameter. The breath signal analysis unit 130 may include a target signal for analysis receiver 131 and a calculator 133. The target signal for analysis receiver 131 may receive the target signal for analysis. The calculator 133 may calculate a volume, a velocity, and the like of the breath flow. In this instance, the diagnosis parameter may include at least one of a peak expiratory flow rate (PEF), a first second forced expiratory volume (FEV 1.0), a forced vital capacity (FVC), and FEV 1.0/FVC. A general calculation scheme may be used as a calculation scheme for a volume, a velocity, and the like of the breath flow.

The display unit 140 may display an analysis result of the breath signal. The display unit 140 may display a result of analysis of the breath signal, or display high/moderate/low of the volume of the breath flow.

The storage unit 150 may store the result of analysis of the breath signal. According to an embodiment, the storage unit 150 may include a mobile storage medium.

The portable spirometer 100 may display, on the display unit 140, data corresponding to a highest lung capacity measurement value in data stored in the storage unit 140 in response to power being turned ON. When measuring of a lung capacity is performed a several times after power is turned ON, the portable spirometer 100 may display a highest value, and may operate in a standby state after a predetermined period of time. According to an embodiment, the portable spirometer 100 may include a processor to control various operations of the portable spirometer 100.

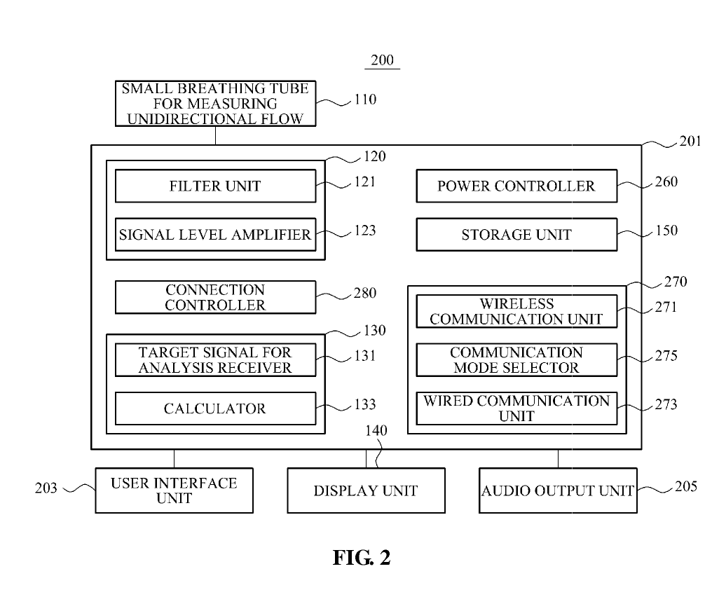

FIG. 2 illustrates a configuration of a portable spirometer according to another embodiment of the present invention.

A portable spirometer 200 illustrated in FIG. 2 may be suitable as a portable type, and may include components for realizing a telemedicine and expending a low amount of power. Reference numbers of FIG. 2 illustrate components performing the same function and operation as reference numbers of FIG. 1. Thus, further descriptions of components having the same reference number as those of FIG. 1 will be omitted for conciseness and ease of description.

The portable spirometer 200 may include all components of the portable spirometer 100, and include a small breathing tube for measuring unidirectional flow 110, a body portion 201, a user interface unit 203, a display unit 140, and an audio output unit 205.

The body portion 201 may include a breath signal processing unit 120, a breath signal analysis unit 130, a power controller 260, a communication unit 270, and a connection controller 280.

In response to the small breathing tube for measuring unidirectional flow 110 being detached, the power controller 260 may block a power supply to the breath signal processing unit 120 and the breath signal analysis unit 130. In response to the small breathing tube for measuring unidirectional flow 110 being detached, the portable spirometer 200 may perform an operation other than a lung capacity measurement. Thus, to reduce power from being wastefully expended, the power controller 260 may block a power supply to the breath signal processing unit 120 and the breath signal analysis unit 130 in response to the small breathing tube for measuring unidirectional flow 110 being detached. The power controller 260 may include a mechanical switch, a transistor, a soft switch, and the like.

The communication unit 270 may exchange data with an external device. That is, the communication unit 270 may transmit data stored in a storage unit 140 to a personal computer (PC), and the like, or transmit a target signal for analysis to an external device. The communication unit 270 may include a wireless communication unit 271, a wired communication unit 273, and a communication mode selector 275.

The wireless communication unit 271 may wirelessly exchange data with an external device. The wireless communication unit 271 may perform wireless communication with a mobile phone, a laptop computer, a PC, and the like using a wireless interface of short distance communication such as Bluetooth communication, infrared-ray communication, a wireless local area network (LAN), and the like.

The wired communication unit 273 may wirelessly exchange data with the external device. To achieve this communication, the wired communication unit 273 may include a connector, a cable connecting terminal, a universal serial bus (USB), and the like.

The communication mode selector 275 may inactivate one of the wireless communication unit 271 and the wired communication unit 273 in response to the same device being connected to the wireless communication unit 271 and the wired communication unit 273. To achieve this communication, the communication mode selector 275 may include a unit to detect whether the same device is connected to the wireless communication unit 271 and the wired communication unit 273, and a unit to connect the portable spirometer 200 and the external device via one of the wireless communication unit 271 and the wired communication unit 273 according to a predetermined scheme in response to the same device being detected to be connected to the wireless communication unit 271 and the wired communication unit 273. In this instance, the predetermined scheme may be determined based on a selection of a user or a communication state. The communication mode selector 275 may determine whether the same device is connected to the wireless communication unit 271 and the wired communication unit 273 using identification (ID) information received from the external device. In response to determining a residual quantity of a battery (not shown) included in the portable spirometer 200 to be inadequate, the communication mode selector 275 may control the communication unit 270 to perform wired communication thus expending less power when compared to wireless communication.

The connection controller 280 may inactivate the breath signal analysis unit, and control so that the target signal for analysis does not pass through the breath signal analysis unit and is transmitted to the external device through the wireless communication unit or the wired communication unit in response to the wireless communication unit or the wired communication unit being connected to the external device. That is, in response to a communication state being set between the portable spirometer 200 and the PC, the connection controller 280 may perform a function for analyzing a breath signal through software installed in the PC, thereby reducing an amount of power expended and performing a relatively accurate measurement.

The user interface 203 may include a button or a keypad to be operated by a user.

The audio output unit 205 may inform a patient that a measurement is completed by outputting a mechanical sound in response to a breath flow being input at an amount greater than or equal to a predetermined amount.

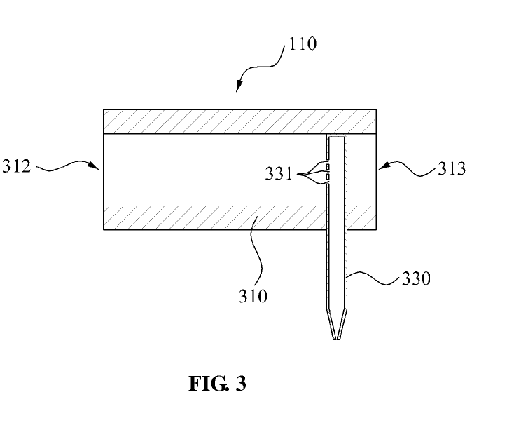

FIG. 3 illustrates a cross-sectional view of a small breath tube for measuring a unidirectional flow of FIG. 1 or FIG. 2.

Referring to FIG. 3, a small breathing tube for measuring a unidirectional flow 110 may be formed by disposable paper or plastic, and may include a circular tube 310 that includes an entrance 312 to be brought into contact with the mouth of a patient, and an outlet 313 opposing the entrance 312, and a sensing path 330 formed to be adjacent to an outlet of the circular tube 310, and formed to have a relatively thin stick type circular tube having an internal diameter of about 1 millimeter (mm).

The sensing path 330 may be formed to be adjacent to the outlet side of the circular tube 310, within a tolerance of about 5 mm, and be formed to have a relatively thin stick type circular tube having an internal diameter of about 1 mm that passes through the circular tube 310 from an upper portion of the circular tube 310 to be extended to a lower portion outside of the circular tube 310. The upper portion of the sensing path 330 may be closed, and the lower portion of the sensing path 330 may be open. Multiple sampling holes 331 for measuring a flow separated by a predetermined interval along a lengthwise direction may be formed at one side of the sensing path 330 formed inside of the circular tube 310, that is, at an entrance side of the circular tube 310.

The circular tube 310 may have a length of about 35 mm and a diameter of about 15 mm, and a fluid resistance may be nearly absent in an inside of the circular tube 310 corresponding to a breathing route of the small breathing tube for measuring a unidirectional flow 110 since only the sensing path 330 corresponding to the relatively thin stick type circular tube having an internal diameter of about 1 mm may be present. A total of three sampling holes 331 formed at one side of the sensing path 330 (that is, the entrance side of the circular tube 310) may be located on a central axis of a flow and at positions apart from the central axis by ±2.5 mm.

A length of the circular tube 310 included in the small breathing tube for measuring a unidirectional flow 110 may be set to 35 mm, which may correspond to a minimum length, so that a patient may breathe easily with the circular tube 310 in a mouth, and the sensing path 330 for measuring a velocity of the flow may be inserted into the circular tube 310. In response to the length of the circular tube 310 being set, a diameter of the circular tube 310 and a location at which the sensing path 330 is formed may be determined according to the set length. In this instance, a diameter of the small breathing tube for measuring a unidirectional flow 110 may be manufactured, so as to satisfy a standard of American Thoracic Society (ATS).

ATS advises that a maximum value of a fluid resistance of a clinical spirometer be about 1.5 cmH2O/Λ/sec, a maximum value of a fluid resistance of a spirometer for self-diagnosis be about 2.5 cmH2O/Λ/sec, and a maximum breath flow value (F) to be measured be about 14 Λ/sec.

The exemplary embodiments according to the present invention may be recorded in computer-readable media including program instructions to implement various operations embodied by a computer. The media may also include, alone or in combination with the program instructions, data files, data structures, and the like. The media and program instructions may be those specially designed and constructed for the purposes of the present invention, or they may be of the well-known variety and available to those having skill in the computer software arts. Examples of computer-readable media include magnetic media such as hard disks, floppy disks, and magnetic tape; optical media such as CD ROM discs and DVD; magneto-optical media such as optical discs; and hardware devices that are specially configured to store and perform program instructions, such as read-only memory (ROM), random access memory (RAM), flash memory, and the like. Examples of program instructions include both machine code, such as produced by a compiler, and files containing higher level code that may be executed by the computer using an interpreter. The described hardware devices may be configured to act as one or more software modules in order to perform the operations of the above-described embodiments of the present invention.

Although a few embodiments of the present invention have been shown and described, the present invention is not limited to the described embodiments. Instead, it would be appreciated by those skilled in the art that changes may be made to these embodiments without departing from the principles and spirit of the invention, the scope of which is defined by the claims and their equivalents.

It is to be understood that both the foregoing general description and the following detailed description are exemplary and explanatory and are intended to provide further explanation of the invention as claimed.

1. A portable spirometer, comprising:
a small breathing tube for measuring a unidirectional flow, to which a breath flow of a patient is inputted;
a breath signal processing unit to generate a breath signal from the breath flow, remove noise contained in the breath signal, and amplify a signal level so as to generate a target signal for analysis;
a breath signal analysis unit to analyze the target signal for analysis to calculate a diagnosis parameter; and
a display unit to display an analysis result of the breath signal.

2. The portable spirometer of claim 1, wherein the small breathing tube for measuring a unidirectional flow comprises:
a circular tube including an entrance, formed by disposable paper or plastic, to be brought into contact with the mouth of the patient, and an outlet opposing the entrance; and
a sensing path formed to be adjacent to the outlet of the circular tube and to pass through the circular tube from an upper portion to be extended to a lower portion outside of the circular tube, and formed to have a tubular shape in which the upper portion is closed and the lower portion is open.

3. The portable spirometer of claim 2, wherein the sensing path has multiple sampling holes for measuring a flow separated by a predetermined interval along a lengthwise direction at an entrance of the circular tube, on a breathing route of the circular tube.

4. The portable spirometer of claim 1, further comprising:
a power controller to block a power supply to the breath signal processing unit and the breath signal analysis unit in response to the small breathing tube for measuring a unidirectional flow being removed.

5. The portable spirometer of claim 1, further comprising:
a wireless communication unit to wirelessly exchange data with an external device;
a wired communication unit to wired-exchange data with the external device; and
a communication mode selector to inactivate one of the wireless communication unit and the wired communication unit in response to the same device being connected to the wireless communication unit and the wired communication unit.

6. The portable spirometer of claim 5, further comprising:
a connection controller to inactivate the breath signal analysis unit, and control so that the target signal for analysis does not pass through the breath signal analysis unit and is transmitted to the external device through the wireless communication unit or the wired communication unit in response to the wireless communication unit or the wired communication unit being connected to the external device.

7. The portable spirometer of claim 1, further comprising:
a storage unit to store the analysis result of the breath signal,
wherein the display unit displays data corresponding to a highest lung capacity measurement value in data stored in the storage unit in response to power being turned ON.

8. The portable spirometer of claim 1, wherein the diagnosis parameter comprises at least one of a peak expiratory flow rate (PEF), a first second forced expiratory volume (FEV 1.0), a forced vital capacity (FVC), and FEV 1.0/FVC.

9. The portable spirometer of claim 1, further comprising:
a power controller to block a power supply to the breath signal processing unit and the breath signal analysis unit in response to the small breathing tube for measuring a unidirectional flow being removed;
a wireless communication unit to wirelessly exchange data with an external device;
a wired communication unit to wired-exchange data with the external device;
a communication mode selector to inactivate one of the wireless communication unit and the wired communication unit in response to the same device being connected to the wireless communication unit and the wired communication unit; and
a storage unit to store the analysis result of the breath signal, wherein the display unit displays data corresponding to a highest lung capacity measurement value in data stored in the storage unit in response to power being turned ON.

10. The portable spirometer of claim 9, wherein the small breathing tube for measuring a unidirectional flow comprises:
a circular tube including an entrance, formed by disposable paper or plastic, to be brought into contact with the mouth of the patient, and an outlet opposing the entrance; and
a sensing path formed to be adjacent to the outlet of the circular tube and to pass through the circular tube from an upper portion to be extended to a lower portion outside of the circular tube, and formed to have a tubular shape in which the upper portion is closed and the lower portion is open.

11. The portable spirometer of claim 9, wherein the sensing path has multiple sampling holes for measuring a flow separated by a predetermined interval along a lengthwise direction at an entrance of the circular tube, on a breathing route of the circular tube.

12. The portable spirometer of claim 9, further comprising:
a connection controller to inactivate the breath signal analysis unit, and control so that the target signal for analysis does not pass through the breath signal analysis unit and is transmitted to the external device through the wireless communication unit or the wired communication unit in response to the wireless communication unit or the wired communication unit being connected to the external device.

13. The portable spirometer of claim 9, wherein the diagnosis parameter comprises at least one of a peak expiratory flow rate (PEF), a first second forced expiratory volume (FEV 1.0), a forced vital capacity (FVC), and FEV 1.0/FVC.
